(19) United States
(12) Patent Application Publication
(10) Pub. No.: US 2011/0288570 A1
(43) Pub. Date: Nov. 24, 2011

(54) ANASTOMOSIS DEVICE

(75) Inventor: Vincent G. Copa, Minnetonka, MN (US)

(73) Assignee: AMS Research Corporation, Minnetonka, MN (US)

(21) Appl. No.: 13/141,046

(22) PCT Filed: Dec. 21, 2009
(86) PCT No.: PCT/US09/69025

§ 371 Date: Jul. 28, 2011

Related U.S. Application Data
(60) Provisional application No. 61/140,120, filed on Dec. 23, 2008.

(51) Int. Cl. A61B 17/11; B29C 65/42

(52) U.S. Cl. 606/153; 156/303.1

(57) ABSTRACT

An anastomosis device for avoiding stretching of a catheter body during an extending healing period while reducing scrap costs. The anastomosis device includes a catheter body defining a proximal end portion and a distal end portion, wherein a device body portion is operably attached at the proximal end portion. The device body portion includes a funnel shaped aperture connecting the device body portion to the proximal end portion. The device body portion provides access into the catheter body so as to allow for placement of a tension cable or flexible core tubing within the catheter body following inspection and verification of an overmold between the device body portion and catheter body. Placement of the tension cable or flexible core tubing subsequent to overmolding reduces scrap costs while eliminating stretching of the catheter body.

### PRIORITY CLAIM

The present application claims priority to U.S. Provisional Application Ser. No. 61/140,120, filed Dec. 23, 2008 and entitled “ANASTOMOSIS DEVICE”, which is herein incorporated by reference in its entirety.

### FIELD OF THE DISCLOSURE

The invention relates generally to anastomosis devices and their associated methods of construction. More particularly, the present application is directed to improved device design and fabrication techniques for anastomosis devices so as to reduce scrap and labor expenses associated with scrap and rework of defectively manufactured products.

### BACKGROUND OF THE DISCLOSURE

Anastomosis procedures are required for connecting or re-connecting certain body tissues, e.g., as part of a surgical procedure. Typically, these tissues define a body lumen such as a blood vessel, intestinal, digestive or urinary tissue that are severed and/or reconnected as part of a successful treatment. One representative example includes a radical prostatectomy procedure in which, a surgeon removes all or most of a patient's prostate. The procedure generally leaves a severed urethral stump and a severed bladder neck, which must be reconnected to so as to restore proper urinary functions.

In response to the inherent risks involved with conventional suturing procedures on such tissue, anastomosis devices have been developed that include a drainage feature and tissue approximating structure that allow for reconnection of tissues without using traditional sutures. Examples of such anastomosis devices are described in U.S. patent publications 2006/0206122A1 and 2006/0264985A1, both of which are herein incorporated by reference in their entirety. These anastomosis devices are commercially available from the owner of the present application, American Medical Systems of Minnetonka, Minn.

Successful use of these anastomosis devices in reconnecting tissue generally requires the devices to remain in position within the patient. Typically the anastomosis device physically retains the reconnected tissue in approximation for a time period ranging from approximately 7 to 21 days or longer. As the anastomosis device remains within the patient for a significant period of time, there is a need for the device to be sufficiently strong and flexible to accommodate the various stresses to which the device may be subjected while positioned within the patient. While it is important that the device remains flexible, it is equally important that the device remain resistant to stretching, which can negatively impact tissue approximation and retention during the healing period.

In order to accommodate the dual requirements of flexibility and stretch-resistance, the aforementioned anastomosis devices have typically utilized a tension cable that is positioned within and bonded to a catheter extrusion prior to attachment of an overmolded funnel. Unfortunately, the overmolding of the funnel to the catheter extrusion is highly subject to fabrication conditions and technique, which can result in a defective connection. As the tension cable is bonded to the catheter extrusion prior to this overmolding process, a defective overmold generally results in the entire assembly being scrapped and discarded.

While the anastomosis devices described above are used to effectively reconnect tissue during surgical procedures, it would be advantageous to improve upon their design so as to provide flexible, stretch-resistant devices that are capable of fabrication with minimal rework and/or scrap of defective assemblies.

### SUMMARY OF THE DISCLOSURE

A representative anastomosis device of the present disclosure accomplishes the dual goals of providing both a flexible, stretch-resistant catheter body while at the same time reducing the cost impact of rework or scrap costs. The anastomosis device can comprise an actuation portion and a catheter portion that are operably connected for performing treatment within a patient. Upon completion of the treatment, the actuation portion can be detached so as to allow the catheter portion to remain in position within the patient during an extended healing period. The catheter portion generally includes a device body portion and a catheter body, with the device body portion operably connected to a proximal end portion of the catheter body. The device body portion can include a funnel shaped aperture that is overmolded to the proximal end portion. In one representative embodiment, the device body portion further includes an insertion aperture providing access to an interior of the catheter body following overmolding of the device body portion and catheter body. A tension cable can be slidingly inserted into the catheter body through the insertion aperture such that the catheter body remains both flexible and stretch-resistant. In another alternative embodiment, the device body portion can include a barbed recess allowing for placement and retention of a length of barbed core tubing. Placement of the tension cable or barbed cored core tubing subsequent to overmolding of the device body portion and catheter body reduces potential scrap costs resulting from a defective overmolding while also eliminating stretching of the catheter body.

In one aspect of the present disclosure, an anastomosis device can comprise a catheter portion having a device body portion and a catheter body for providing minimally invasive procedures within a patient. The catheter body generally defines a continuous lumen between a proximal end portion and a distal end portion. The device body portion includes a funnel-shaped aperture for operably overmolding the device body portion to the catheter body to define the catheter portion. The device body portion includes an insertion aperture allowing for exterior access to the catheter body such that a tension cable can be slidingly inserted into the catheter body. Placement of the tension cable into the catheter body through the insertion aperture allows the catheter body to remain both flexible and stretch-resistant while reducing any cost impact due to scrap or rework resulting from a defective overmold between the device body portion and the catheter body.

In another aspect of the present disclosure, an anastomosis device can comprise a catheter portion having a device body portion and a catheter body for providing minimally invasive procedures within a patient. The catheter body generally defines a continuous lumen between a proximal end portion and a distal end portion. The device body portion includes a funnel-shaped aperture for operably overmolding the device body portion to the catheter body so as to define the catheter portion. The device body portion further includes a barbed recess proximate the funnel-shaped aperture allowing for the insertion and retention of a length of barbed core tubing within the catheter body. Placement and retention of the core tubing within the catheter body allows the catheter body to remain both flexible and stretch-resistant while allowing the core tubing to be inserted only after verification of a successful overmold between the device body portion and the catheter body. By verifying that a successful overmold has been accomplished prior to placing the core tubing, scrap and rework costs can be drastically reduced.

In another aspect of the disclosure, an anastomosis device can be fabricated by providing a device body portion with a funnel shaped aperture, access ports and an insertion aperture. The device body portion can be joined to a catheter body through overmolding of the funnel shaped aperture and a proximal end portion of the catheter body. The joined device body portion and catheter body is inspected immediately following the overmolding process so as to identify defective connections requiring rework or scrapping of the joined device body portion and catheter body. Following inspection of the overmold, a tension cable can be slidingly inserted through the insertion aperture and into the catheter body so as to eliminate stretching of the catheter body when positioned within a patient.

In yet another aspect of the disclosure, scrap and rework costs associated with fabrication of an anastomosis device can be reduced by inserting a tension cable or length of core tubing only after inspection and verification of a satisfactory overmold connection between a catheter body and a device body portion.

The above summary of the invention is not intended to describe each illustrated embodiment or every implementation of the present invention. The Figures and the detailed description that follow more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 is a partially hidden, perspective view of an anastomosis device of the prior art.

FIG. 2 is an exploded, perspective view of a catheter portion of an anastomosis device according to an embodiment of the present disclosure.

FIG. 3 is a partially hidden, side view of a catheter portion of an anastomosis device according to an embodiment of the present disclosure.

FIG. 4 is a side view of a length of core tubing for use with the catheter portion of FIG. 3.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE FIGURES

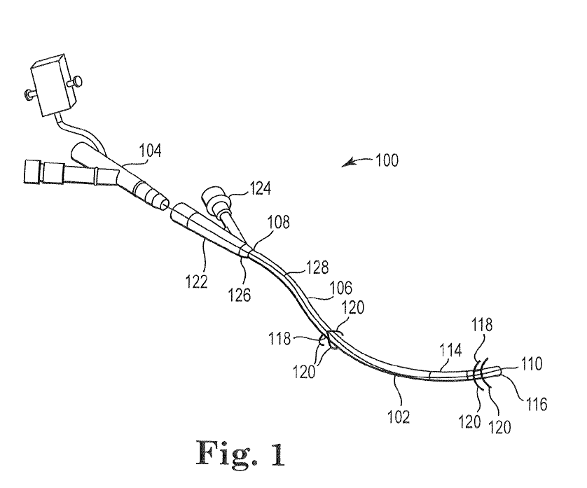

As illustrated in FIG. 1, a representative embodiment of an anastomosis device 100 can comprise a catheter portion 102 and an actuation portion 104. Catheter portion 102 generally comprises a catheter body 106 having a proximal end portion 108 and a distal end portion 110. Located proximate to distal end portion 110, the catheter body can include an inflation balloon 114, a drainage aperture 116 and a tissue approximating structure 118. Tissue approximating structure 118 can comprise a single structure or multiple structures generally positioned along the catheter body 106 between the distal end portion 110 and the proximal end portion 108. With the exception of the tissue approximation structure 118, anastomosis device 100 can physically resemble the structure of a conventional Foley catheter that is typically inserted into a patient's bladder to drain urine.

As shown in FIG. 1, tissue approximating structure 118 can include multiple sharp elongate tines 120 that can extend from and retract into catheter body 106 at a location that allows contact and optional penetration of adjacent tissue structures. For example, the tissue approximating structure 118 can be positioned within a patient to allow contact and optional penetration of any of the aforementioned body lumens including, but not limited to, blood vessels, intestinal, digestive, reproductive or urinary tissue that are to reconnected to establish a continuous lumen. In some embodiments, tissue approximating structure 118 can specifically include two sets of elongated structures, which are deployable or otherwise moveable in opposed directions from each other. The elongated structures can be rigid or semi-rigid tines, needles, or the like, having a straight or curved configuration. In some embodiments, the elongated structures can include a sharp pointed tip to penetrate into a body lumen or other tissue that can be brought into contact and/or held together during a tissue healing period. In some embodiments, the tissue approximating structure can include both a distal set of tissue approximating structures and a proximal set of tissue approximating structures located on opposite sides of inflation balloon 114, wherein both sets can be controlled simultaneously or individually depending upon the specific treatment protocol.

Referring again to FIG. 1, proximal end portion 108 of the anastomosis device 100 can comprise a device body portion 122 that extends from proximal end portion 108. Device body portion 122 generally possesses an increased diameter than catheter body 106. Device body portion 122 can include one or more ports 124 that extend or otherwise protrude from the device body portion 122 and generally provide access to a lumen (not shown) defined within the catheter body 106 that can provide for inflation of the balloon 114 or for drainage from the drainage aperture 116. Device body portion 122 generally includes a funnel shaped aperture 126 configured for operable connection to the proximal end portion 108 using an overmolding procedure so as to establish a leak-free seal between the catheter body 106 and the device body portion 122.

Catheter body 106 is generally constructed of a soft plastic or rubber tube. In using anastomosis device 100, distal end portion 110 is slidably advanced to a treatment site wherein the tissue approximation structure 118 can be deployed so as to capture, retain and approximate tissue during a healing period. The healing period can last anywhere from 7 to 21 days or longer, depending upon the procedure and tissue type. With the tissue approximation structure 118 deployed during this healing period, it is critical that stretching of the catheter body 106 be avoided even as the anastomosis device 100 is exposed to everyday forces and stresses generated by the body and its associated activities.

As shown in FIG. 1, one method by which stretching of the catheter body 106 can be avoided is through the bonding of a tension cable 128 within the length of catheter body 106. Tension cable 128 is typically fabricated of a non-stretch or stretch resistant material such as, for example, a medically suitable stainless steel. Tension cable 128 is generally bonded to catheter body 106 prior to attachment of the device body portion 122 to the proximal end portion 108. As such, a failure or defect within the overmolding connection of the funnel shaped aperture 126 and the proximal end portion 108 requires the tension cable 128 to be discarded as the bonding process makes the tension cable 128 integral to the catheter body 106 and correspondingly, the defective anastomosis device 100.

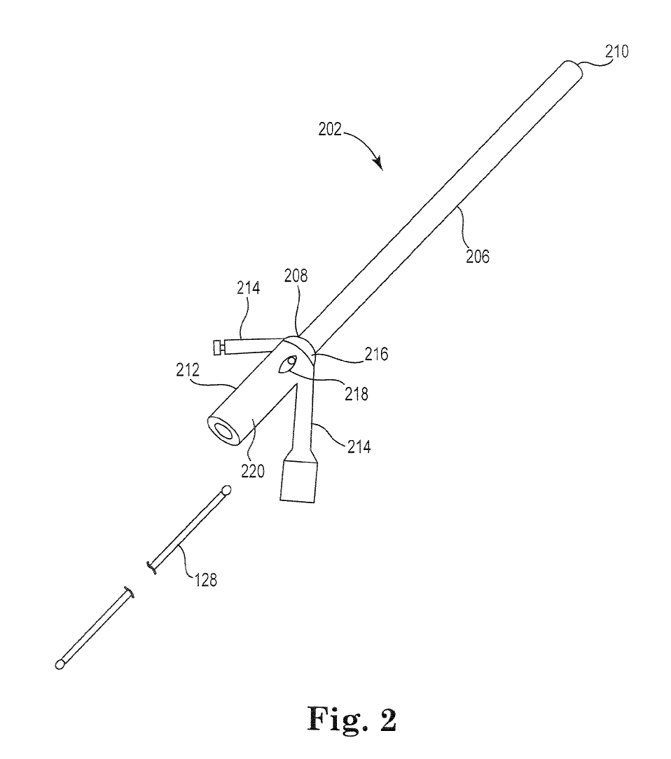

Referring to FIG. 2, an embodiment of a catheter portion 202 for use with actuation portion 104 can comprise a catheter body 206 having a proximal end portion 208 and a distal end portion 210 with a lumen (not pictured) extending between the proximal end portion 208 and distal end portion 210 defined therein. The lumen can provide, for example, for inflation of a balloon or for drainage from a drainage aperture. When catheter portion 202 is connected to actuation portion 104, the combination can substantially resemble anastomosis device 100 in both appearance and materials of construction. At proximal end portion 208, a device body portion 212 is operably attached so as to provide access to the catheter body 206 through one or more ports 214. Device body portion 212 generally possesses an increased diameter than catheter body 106 and includes a funnel shaped aperture 216 configured for operable connection to the proximal end portion 208 using an overmolding procedure so as to establish a leak-free seal between the catheter body 206 and the device body portion 212. Device body portion 216 further comprises an insertion aperture 218 providing access from an exterior surface 220 of the device body portion 216 into the funnel shaped aperture 216, and correspondingly into the catheter body 206 and associated lumen. Insertion aperture 218 generally defines a diameter slightly increased in comparison to the tension cable 128.

As described previously with respect to anastomosis device 100, catheter body 206 is generally constructed of a soft plastic or rubber tube and capable of being inserted to a treatment site and remaining during a healing period. In fabricating anastomosis device 200, the presence of insertion aperture 218 on device body portion 212 allows the overmolding of device body portion 212 and catheter body 206 to be performed prior to inserting and bonding tension cable 128 within the catheter body 206. Generally, funnel shaped aperture 216 is positioned relative to proximal end portion 208 such that the overmolding process can be performed. Following the joining of device body portion 212 and catheter body 206, the integrity of the overmold joint at the junction of the funnel shaped aperture 216 and proximal end portion 208 can be inspected and verified. Following this inspection, assemblies that satisfy the test criteria can have the tension cable 128 slidingly inserted into the device body portion 212 through the insertion aperture 218. The tension cable 128 is then advanced through the funnel shaped aperture 216, into the proximal end portion 208 and through the length of the catheter body 206 until the tension cable 128 is proximate the distal end portion 210. The tension cable 128 can be inserted into the previously described lumen through catheter body 206 or can be inserted through a separate cable lumen. The tension cable 128 can then be bonded to the catheter body 206 and the insertion aperture 218 filled such that stretching of the catheter portion 202 is avoided as the anastomosis device is exposed to everyday forces and stresses encountered during the healing period in which the catheter body 206 remains in place within the body.

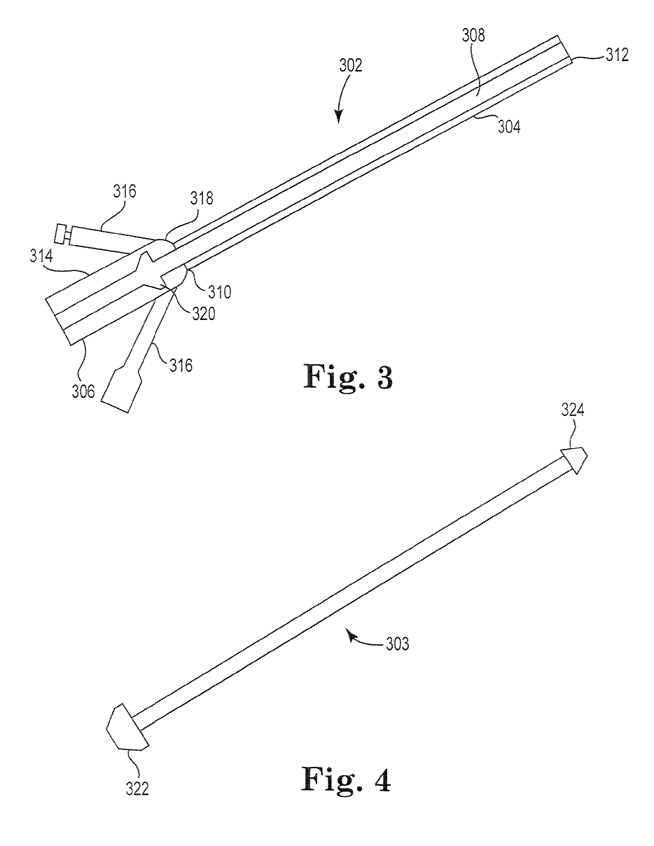

In another representative embodiment of an anastomosis device, elimination of rework and scrap expense can be accomplished through substitution of a length of core tubing for the tension cable 128. Generally, an anastomosis device can comprise a catheter body 302 and a length of core tubing 303 as shown in FIGS. 3 and 4. Catheter body 302 generally includes a catheter portion 304 for operable connection to actuation portion 104 such that the anastomosis device generally resembles anastomosis device 100 in both appearance and materials of construction. Catheter portion 304 generally comprises a catheter shaft 308, or lumen, having a proximal end portion 310 and a distal end portion 312. At proximal end portion 310, a device body portion 314 is operably attached so as to provide access to the catheter shaft 308 using one or more ports 316. Device body portion 314 generally possesses an increased diameter than catheter shaft 308 and includes a funnel shaped aperture 318 configured for operable connection to the proximal end portion 310 using an overmolding procedure so as to establish a leak-free seal between the catheter shaft 308 and the device body portion 314. Device body portion 314 further comprises a barbed receiver 320 within the funnel shaped aperture 318.

Core tubing 303 generally includes a barbed proximal end 322 and a barbed distal end 324. Core tubing 303 can be fabricated of a suitable medical polymer such as, for example, low density polyethylene. Barbed proximal end 322 is configured so as to substantially resemble the size and shape of barbed receiver 320. Generally, barbed distal end 324 can be slidingly inserted into one of ports 316 in the device body portion 314 such that barbed distal end 324 enters the catheter shaft 308 through the funnel shaped aperture 318. As the barbed distal end 324 approaches the distal end portion 312, barbed proximal end 322 snaps into positioned within barbed receiver 320 such that core tubing 303 is fixedly positioned and retained within the catheter body 302.

With core tubing 303 retained within catheter body 302, stretching of the catheter shaft 308 can be prevented while still providing flexibility to the catheter body 302. In addition, insertion of the core tubing 303 is performed only after a successful overmold is accomplished between the catheter shaft 308 and the device body portion 314. As such, rework or scrap expenses are reduced as core tubing 303 is installed only after inspection of the overmold.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

1. An anastomosis device comprising:
a catheter body portion having a proximal end and a distal end and defining a body lumen extending therethrough from the proximal end to the distal end;
a device body portion overmolded to the proximal end of the catheter portion, the device body portion having an exterior surface including an insertion aperture providing access to a device lumen in the device body portion; and
a tension cable slidably inserted though the insertion aperture following verification of joint integrity between the catheter body and the device body portion, the tension cable entering the device lumen such that the tension cable can be advanced into the body lumen, wherein the tension cable is bonded within the catheter body portion and the device body portion such that the insertion aperture is filled and no longer provides access to the device lumen.

2. The anastomosis device of claim 1, wherein the device body portion includes a funnel-shaped portion, and wherein the proximal end of the catheter portion is overmolded to the funnel-shaped portion.

3. (canceled)

4. A method for fabricating an anastomosis device to reduce rework and scrap costs, comprising:
providing a catheter body portion having a proximal end and a distal end and defining a body lumen extending therethrough from the proximal end to the distal end;
providing a device body portion including a device lumen and having an insertion aperture in an exterior surface, wherein the insertion aperture is fluidly interconnected to the device lumen;
overmolding the proximal end of the catheter body portion to the device body portion such that the body lumen and device lumen are fluidly interconnected;
inspecting and verifying the integrity of the overmold connection between the catheter body portion and the device body portion;
inserting a tension cable through the insertion aperture after the integrity of the overmold connection between the catheter body portion and the device body portion as been verified, the tension cable entering into the device lumen and advancing into the body lumen; and
bonding the tension cable within the catheter body portion and device body portion.

5. (canceled)

6. The method of claim 4, wherein the catheter body portion includes a cable lumen extending between the proximal end and the distal end and wherein the step of inserting the tension cable through the insertion aperture and into the catheter body includes inserting the tension cable into the device lumen and into the cable lumen.

7. An anastomosis device comprising:
a catheter shaft having a proximal end and a distal end and defining a body lumen extending therethrough from the proximal end to the distal end;
a device body portion overmolded to the proximal end of the catheter shaft, the device body portion having a device lumen fluidly connected to the body lumen, wherein the device lumen defines a receiver portion, and wherein the device body portion includes one or more ports providing access into the device lumen; and
a length of core tubing having a proximal tube end and a distal tube end, wherein slidable advancement of the core tubing into the one or more ports results in the core tubing residing with the body lumen and the device lumen with a tube receiver at the distal tube end fixedly retained within the receiver portion.

8. The anastomosis device of claim 7, wherein the core tubing further includes a barbed distal end.

9. The anastomosis device of claim 7, wherein the receiver portion defines a funnel shaped aperture and wherein the tube receiver has a receiver profile adapted for retention with the funnel shaped aperture.

10. A method for fabricating an anastomosis device to reduce rework and scrap costs, comprising:
providing a catheter body portion having a proximal end and a distal end and defining a body lumen extending therethrough from the proximal end to the distal end;
providing a device body portion including a device lumen, wherein the device lumen defines a receiver portion, and wherein the device body portion includes one or more ports providing access into the device lumen
overmolding the proximal end of the catheter body portion to the device body portion such that the body lumen and device lumen are fluidly interconnected; and
advancing a length of core tubing into the one or more ports such that a tube receiver at a distal tube end of the core tubing is fixedly retained within the receiver portion.

11. The method of claim 10, further comprising:
inspecting the integrity of an overmold connection of the catheter body portion and the device body portion.

12. The method of claim 4, wherein bonding the tension cable within the catheter body portion further comprises:
filling the insertion aperture to permanently couple the tension cable within the catheter body portion and the device body portion.
